(19) United States
(12) Patent Application Publication
(10) Pub. No.: US 2017/0261499 A1
(43) Pub. Date: Sep. 14, 2017

(54) Method for the Aptamer Detection of Multiple Small Molecules of Similar Structure Through Deconvolution

(71) Applicant: DIAGNOSTIC BIOCHIPS, INC., Glen Burnie, MD (US)

(72) Inventor: Emma Bigelow, Baltimore, MD (US)

(73) Assignee: DIAGNOSTIC BIOCHIPS, INC., Glen Burnie, MD (US)

(21) Appl. No.: 15/529,608

(22) PCT Filed: Nov. 25, 2015
(86) PCT No.: PCT/US15/62695

§ 371 Date: May 25, 2017

Related U.S. Application Data
(60) Provisional application No. 62/084,179, filed on Nov. 25, 2014.

(51) Int. Cl. G01N 33/53; G01N 33/543

(52) U.S. Cl. CPC G01N 33/5308; G01N 33/54306

(57) ABSTRACT

Provided herein are aptamer arrays useful to detect target molecules that are largely similar to each other in structure (such as monoamine neurotransmitters), and methods of detecting such molecules, employing aptamers with differing sensitivities to each target molecule.

### BACKGROUND

Physicians order blood and urine tests, biopsies and other tissue samples that give information about biochemical concentrations at one instant in time, however there is almost no information available about how these concentrations vary with time. In the case of stroke or heart attack, for example, it has been shown that the presence of certain biomarkers indicates a high probability of onset, however there is no way to practically monitor an at-risk patient for the sudden appearance of these markers.

Existing approaches for diagnostics of target molecules (chemicals of interest) require carefully-engineered sensing elements that are difficult to tailor to a particular application.

Aptamers are nucleic acid molecules having specific binding affinity to molecules through interactions other than classic Watson-Crick base pairing.

Aptamers, like peptides generated by phage display or monoclonal antibodies (“mAbs”), are capable of specifically binding to selected targets and modulating the target's activity, e.g., through binding aptamers may block their target's ability to function. Created by an in vitro selection process from pools of random sequence oligonucleotides, aptamers and SOMAmers® (“Slow Off-rate Modified Aptamers”) have been generated for over 3000 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., aptamers will typically not bind other proteins from the same gene family). A series of structural studies have shown that aptamers are capable of using the same types of binding interactions (e.g., hydrogen bonding, electrostatic complementarity, hydrophobic contacts, and steric exclusion) that drive affinity and specificity in antibody-antigen complexes.

Aptamers have a number of desirable characteristics for use as therapeutics and diagnostics including high specificity and affinity, biological efficacy, and excellent pharmacokinetic properties.

The similarities between many neurotransmitters can make it difficult to select an aptamer that is sensitive and highly specific to a particular neurotransmitter or other potential target of similar structure, including proteins that are in the same family and have some analogous structural features. Instead of re-selecting aptamers over and over aiming for high specificity against one target and excluding sensitivity to others, we could benefit from the cross-reactivity of aptamers to collect information on multiple neurotransmitters while also improving the overall specificity of the sensor.

### SUMMARY

Provided herein are aptamer arrays useful to detect molecules that are largely similar to each other in structure (such as monoamine neurotransmitters).

### DETAILED DESCRIPTION

As used herein, the term “aptamer” or “specifically binding oligonucleotide” refers to an oligonucleotide that is capable of forming a complex with an intended target substance. The complexation is target-specific in the sense that other materials which may accompany the target do not complex to the aptamer. It is recognized that complexation and affinity are a matter of degree; however, in this context, “target-specific” means that the aptamer binds to target with a much higher degree of affinity than it binds to contaminating materials.

Generally, aptamers are macromolecules composed of nucleic acid, such as RNA or DNA that bind tightly to a specific molecular target. As is typical of nucleic acids, a particular aptamer may be described by a linear sequence of nucleotides (A, U or T, C and G). These sequences are generally about 15-60 bases long. In practice, however, the chain of nucleotides forms intramolecular interactions that result in a molecule with a complex three-dimensional shape. The shape of the aptamer contributes to its ability to bind tightly against with surface of its target molecule. Since a tremendous range of molecular shapes exist among the possibilities for nucleotide sequences, aptamers may be obtained for a wide array of molecular targets, including most proteins and many small molecules.

The aptamer may be prepared by any known method, including synthetic, recombinant, and purification methods, and may be used alone or in combination with other aptamers specific for the same target. Further, as described more fully herein, the term “aptamer” specifically includes “secondary aptamers” containing a consensus sequence derived from comparing two or more known aptamers to a given target.

### Arrays

The arrays described herein allows the use of aptamers to detect molecules that are largely similar to each other in structure (such as monoamine neurotransmitters), that was previously limited by how specific an aptamer could be made for a single target molecule. “Target molecule” or “target” means any compound of interest for which a ligand is desired. A target molecule can be a protein, peptide, carbohydrate, polysaccharide, glycoprotein, hormone, receptor, antigen, antibody, virus, substrate, metabolite, transition state analog, cofactor, inhibitor, drug, dye, nutrient, growth factor, etc., without limitation.

Specific examples of targets that can usefully be detected using the methods and systems described herein include, but are not limited to:
small molecules that have similar structures, such as neurotransmitters GABA and acetylcholine;
other small molecules that bind the same receptor site, such as drug molecules that target the same receptor;
proteins that require differentiation between proteins in the same family, e.g. proteins that can include two different monomers that create a dimer, such as PDGF-BB (vs. PDGF-AB or PDGF-AA).

Another example of similar proteins that may require signal deconvolution by the described method to resolve individual target concentrations are interleukins-1alpha and -1beta. These proteins share a similarly arranged 12-stranded beta-sheet structure. IL-1beta has been implicated in some cytokine storm clinical studies, and therefore measurement of IL-1beta exclusively may be of interest.

A separate example that relates to monitoring the emergence of a cytokine storm is the measurement of interleukin-6, as distinct from myelomonocytic growth factor (MGF) and granulocyte colony-stimulating factor (GCSF). These three proteins each have compact, globular fold structures, similar to other interleukins. Each protein also has a 4-alpha-helix bundle with a left-handed twist that dominates a significant part of each of the structures. So, if an aptamer was selected for one of these targets but involves binding to that portion of the structure, the aptamer could display some affinity for the other two molecules.

“Oligomers” or “oligonucleotides” include RNA or DNA sequences of more than one nucleotide in either single chain or duplex form and specifically includes short sequences such as dimers and trimers, in either single chain or duplex form, which may be intermediates in the production of the specifically binding oligonucleotides. “Nucleic acids”, as used herein, refers to RNA or DNA sequences of any length in single-stranded or duplex form.

An “array,” “macroarray” or “microarray” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports. The array could either be a macroarray or a microarray, depending on the size of the sample spots on the array. A macroarray generally contains sample spot sizes of about 300 microns or larger and can be easily imaged by gel and blot scanners. A microarray could generally contain spot sizes of less than 300 microns.

“Solid support,” “support,” and “substrate” refer to a material or group of materials having a rigid or semi-rigid surface or surfaces. In some aspects, at least one surface of the solid support could be substantially flat, although in some aspects it may be desirable to physically separate synthesis regions for different molecules with, for example, wells, raised regions, pins, etched trenches, or the like. In certain aspects, the solid support(s) could take the form of beads, resins, gels, microspheres, or other geometric configurations.

In some cases, it may be beneficial to pair aptamers that have different sensitivities for the same target(s). For example, this could either include an aptamer that is sensitive for the low nanomolar range of GABA and an aptamer that is sensitive to the low micromolar range of GABA. In combining these two aptamers, we extend the detectable range of the neurotransmitter.

In another case, specificity can be improved by combining two aptamers that are both sensitive (to different degrees) to two different target molecules. For example, the neurotransmitters GABA and acetylcholine have similar structures. We have an aptamer that has a Kd=˜4 nM to GABA and Kd=˜40 nM to acetylcholine. |If we select an additional sensor that has different sensitivities—for example, more sensitive to acetylcholine than GABA—we can use both aptamers simultaneously to detect both target molecules. |[EB1]By having two aptamers with known (different) sensitivities to two targets, we can essentially deconvolve the two target concentrations by having two equations and two unknowns (unknowns being the target concentrations). This principle can be extended to other classes of proteins with similar structures. For example, TGF-β1 is 71% and 77% similar to TGF-β2 and TGF-β3, respectively.

Such a detecting system may allow for the most sensitive and specific detection of small molecules to which aptamer specificity is a challenge. Other monoamine neurotransmitters are likely to have similar sensitivities as our GABA/acetylcholine aptamer, and therefore this detection scheme could be used for a variety of pairs of neuromodulators.

Even in cases where more specific aptamers are available, it may be advantageous to use a less specific aptamer in the case that: 1) it has more advantageous binding kinetics (i.e. faster signal response), or 2) it is being used to extend the dynamic range of target detection and lower affinity may be accompanied by lower specificity, or 3) this method can be used to use aptamers that increase sensor signal-to-noise ratio, or other sensor characteristics.

The method described herein for the detection of two targets, denominated “X” and “Y,” requires the following components:

One aptamer sequence (1) that has sensitivity to both X and Y. The sensitivity to X and Y will vary; sensitivity to X and Y may vary by >1 order of magnitude; in an alternative embodiment, sensitivity to X and Y will vary by at least 2 orders of magnitude.
A second aptamer sequence (2) that is sensitive to both X and Y, but with differing Kd values (sensitivities) to the two targets. In an alternative embodiment, the sensitivities of the second aptamer sequence is “flipped” from that of the first aptamer sequence (i.e., if the first aptamer was most sensitive to X, then the second aptamer would be most sensitive to Y)
An electrode array onto which both aptamers are bound (to form aptamer-1 sites and aptamer-2 sites). These sites would ideally be spatially close (<40 μm apart) in order to detect from a very localized area. In one alternative embodiment, the aptamers are each applied to a different electrode.

Synthesis of aptamers is well known in the art; any art-known method of synthesizing aptamers may be used to produce aptamers for use in the arrays described herein. Aptamers may be labeled with a detectable label. Many detectable labels are known in the art, and can be selected by the skilled artisan. In constructing the arrays described herein, the aptamers may be bound to the solid support.

### Methods

Also provided herein is method for detecting a first target A and a second target B. The targets may be proteins, peptides, carbohydrates, polysaccharides, glycoproteins, hormones, receptors, antigens, antibodies, viruses, substrates, metabolites, transition state analogs, cofactors, inhibitors, drugs, dyes, nutrients, or growth factors. The method comprises providing a first aptamer α1 and a second aptamer α2 bound to a solid support, wherein the first aptamer is selected to have a sensitivity to the first target of Kd_{A}α₁ which is >0 and a sensitivity to the second target of Kd_{B}α₁ which is >0, and the second aptamer is selected to have a sensitivity to the first target of Kd_{A}α₂ which is >0 and a sensitivity to the second target of Kd_{B}α₂ which is >0, wherein Kd_{A}α₁ differs from Kd_{A}α₂, and Kd_{B}α₁ differs from Kd_{B}α₂.

Kd_{A}α₁ may differ from Kd_{B}α₁ by at least one order of magnitude, alternatively by at least two orders of magnitude. Kd_{B}α₁ may differ from Kd_{B}α₂ by at least one order of magnitude, alternatively by at least two orders of magnitude. Kd_{A}α₁ will optionally be greater than Kd_{B}α₁; in such cases, Kd_{B}α₂ may be greater than Kd_{A}α₂. Similarly, Kd_{A}α₂ will optionally be greater than Kd_{B}α₂; in such cases, Kd_{A}α₁ may be greater than Kd_{B}α₁.

In the method disclosed herein, the aptamers are bound to a single support. In such cases, the first aptamer α₁ is optionally bound to the support at an Aptamer-1 site, and the second aptamer α₂ is bound to the support at an Aptamer-2 site. In one alternative embodiment, the Aptamer-1 site is less than 40 μm from the Aptamer-2 site. Alternatively, the first aptamer α₁ is bound to a first solid support and the second aptamer α₂ is bound to a second solid support.

This method allows for improved specificity of an aptamer-based sensor for small molecules by collecting signals from multiple aptamers with different specificities and integrating for an improved signal.

This method may, of course, be extrapolated to detect more than two targets.

### Deconvolution

The equation for deconvolving the signal attributed to two different targets that both bind to the same aptamer is derived from the Langmuir adsorption model for competitive binding

Assumptions for these equations are:

1) The maximum possible signal for a given aptamer to a given target is highly dependent on experimental conditions, and so must be calibrated for the conditions in which the measurements will occur.
2) There are no inter-target interactions once bound to the aptamers
3) An aptamer can only bind one target molecule at a time
4) All aptamer binding sites are equivalent (and equally exposed to target molecules)

In the equations set forth below, the variables shown have the following meanings:

[A]: concentration of target molecule A
[B]: concentration of target molecule B
[SA]: concentration of molecule A bound to aptamer complex
[SB]: concentration of molecule B bound to aptamer complex
[S]: concentration of available aptamer binding sites (unbound)
[Sₜₒₜₐₗ]: total aptamer binding sites (bound+unbound)
f_{A}: fraction of aptamers bound with target molecule A

r_{adsorption}=kₒₙ [A][S]

r_{desorption}=k_{off} [SA

Langmuir binding for a single molecule A to aptamer site S:

k_{eq,A}=(kₒₙ/k_{off})=[SA]/[A]/[A][S]

[Sₜₒₜₐₗ]=[S]+[SA]

[Sₜₒₜₐₗ]=[SA]/[A]k_{eq,A}+[SA]

f_{A}=[SA]/[Sₜₒₜₐₗ]=[A]/(k_{d,A}+[A])

Langmuir binding for two competitive molecules A, B that can bind to the same site, S:

k_{eq,A}=k_{on,A}/k_{off,A}=[SA]/[A][S]

k_{eq,B}=k_{on,B}/k_{off,B}=[SB]/[B][S]

[Sₜₒₜₐₗ]=[S]+[SA]+[SB]

[Sₜₒₜₐₗ]=[S] (1+k_{eq,A}[A]+k_{eq,B}[B])

f_{A}=[SA]/[Sₜₒₜₐₗ]=[SA]/([S] (1+k_{eq,A}[A]+k_{eq,B}[B]))

f_{A}=k_{eq,A}[A]/(1+k_{eq,A}[A]+k_{eq,B}[B])

f_{B}=k_{eq,B}[B]/(1+k_{eq,A}[A]+k_{eq,B}[B])

Multiply f_{A} and f_{B} by (k_{off,A}/k_{on,A})/(k_{off,A}/k_{on,A}) and (k_{off,B}/k_{on,B})/(k_{off,B}/k_{on,B}), respectively:

f_{A}=[A]/(k_{d,A}+[A]+(k_{d,A}/k_{d,B})[B])

f_{B}=[B]/(k_{d,B}+[B]+(k_{d,B}/k_{d,A})[A])

(k_{d}=1/k_{eq}=k_{off}/kₒₙ)

To convert these equations to calculate the signal generated from aptamer biosensors, we first look at the case where a single molecule A binds the target and generates signal X (and Xₘₐₓ is the maximum signal achieved by A binding aptamer X under given experimental conditions):

X/Xₘₐₓ=[SA]/[Sₜₒₜₐₗ]=[A]/(k_{d,A}+[A])

When [A]=k_{d,A}, X=½ (Xₘₐₓ) as expected by the definition of k_{d}. When [A]>>k_{d,A}, this equation goes to 1, or Xₘₐₓ is achieved.

This same principle is used to convert equations for f_{A} and f_{B} into aptamer sensor signal. In the case where a single aptamer is sensitive to two target molecules, A and B, different Xₘₐₓ values must be applied for each target molecule due to the differing conformational change that may occur when binding to the two different molecules. X_{max,A} and X_{max,B} can be determined experimentally for a given set of experimental conditions. These values can be strongly influenced by ionic content of the test environment, pH, and the presence of divalent cations, such as Mg⁺² and Ca⁺², which stabilize DNA tertiary structures:

X=X_{max,A} ([A]/(k_{d X,A}+[A]+(k_{d X,A}/k_{d X,B})[B]))+X_{max,B} ([B]/(k_{d X,B}+[B]+(k_{d X,B}/ k_{d X,A})[A]))

The above equation has two unknowns—[A] and [B]. A second aptamer, with k_{d,A} and k_{d,B} values differing from the first aptamer, can be used in the same experiment to solve for concentrations of both target molecules:

Y=Y_{max,A} ([A]/(k_{d Y,A}+[A]+(k_{d Y,A}/k_{d Y,B})[B]))+Y_{max,B} ([B]/(k_{d Y,B}+[B]+(k_{d Y,B}/k_{d Y,A})[A]))

These equations can be extended to the case in which 3 aptamers each bind 3 targets with differing affinities (or really any X aptamers with differing affinities to X targets). Additionally, it is possible that one of the aptamers included in such a set may have a very high affinity to one target and effectively no sensitivity to the other targets. This aptamer would still be useful in this method of data analysis. Note: k_{d X,A} indicates the k_{d} values (dissociation constant; =k_{off}/kₒₙ) for aptamer X binding target molecules A.

What is claimed is:

1. A device for the detection of a first target A and a second target B, comprising
a first aptamer α1 bound to a solid support and a second aptamer α2 bound to a solid support, wherein
the first aptamer has a sensitivity to the first target of Kd_{A}α₁ which is >0 and a sensitivity to the second target of Kd_{B}α₁ which is >0, and
the second aptamer with a sensitivity to the first target of Kd_{A}α₂ which is >0 and a sensitivity to the second target of Kd_{B}α₂ which is >0,
wherein Kd_{A}α₁ differs from Kd_{A}α₂, and Kd_{B}α₁ differs from Kd_{B}α₂.

2. The device of claim 1, wherein Kd_{A}α₁ differs from Kd_{B}α₁ by at least one order of magnitude.

3. The device of claim 2, wherein Kd_{A}α₁ differs from Kd_{B}α₁ by at least two orders of magnitude.

4. The device of claim 1, wherein Kd_{B}α₁ differs from Kd_{B}α₂ by at least one order of magnitude.

5. The device of claim 4, wherein Kd_{B}α₁ differs from Kd_{B}α₂ by at least two orders of magnitude.

6. The device of claim 1, wherein Kd_{A}α₁>Kd_{B}α₁.

7. The device of claim 6, wherein Kd_{B}α₂>Kd_{A}α₂.

8. The device of claim 1, wherein Kd_{A}α₂>Kd_{B}α₂.

9. The device of claim 8, wherein Kd_{A}α₁>Kd_{B}α₁.

10. The device of claim 1, wherein the first aptamer αₗ is bound to the support at an Aptamer-1 site, and the second aptamer α₂ is bound to the support at an Aptamer-2 site.

11. The device of claim 10, wherein the Aptamer-1 site is <40 μm from the Aptamer-2 site.

12. The device of claim 1, wherein the targets are selected from the group consisting of proteins, peptides, carbohydrates, polysaccharides, glycoproteins, hormones, receptors, antigens, antibodies, viruses, substrates, metabolites, transition state analogs, cofactors, inhibitors, drugs, dyes, nutrients, and growth factors.

13. The device of claim 1, wherein the first aptamer α₁ is bound to a first solid support and the second aptamer α₂ is bound to a second solid support.

14. A method for detecting a first target A and a second target B, comprising
providing a first aptamer α1 bound to a solid support and a second aptamer α2 bound to a solid support, wherein
the first aptamer is selected to have a sensitivity to the first target of Kd_{A}α₁ which is >0 and a sensitivity to the second target of Kd_{B}α₁ which is >0, and
the second aptamer is selected to have a sensitivity to the first target of Kd_{A}α₂ which is >0 and a sensitivity to the second target of Kd_{B}α₂ which is >0,
wherein Kd_{A}α₁ differs from Kd_{A}α₂, and Kd_{B}α₁ differs from Kd_{B}α₂.

15. The method of claim 14, wherein Kd_{A}α₁ differs from Kd_{B}α₁ by at least one order of magnitude.

16. The device of claim 15, wherein Kd_{A}α₁ differs from Kd_{B}α₁ by at least two orders of magnitude.

17. The method of claim 14, wherein Kd_{B}α₁ differs from Kd_{B}α₂ by at least one order of magnitude.

18. The method of claim 17, wherein Kd_{B}α₁ differs from Kd_{B}α₂ by at least two orders of magnitude.

19. The method of claim 14, wherein Kd_{A}α₁>Kd_{B}α₁.

20. The method of claim 19, wherein Kd_{B}α₂>Kd_{A}α₂.

21. The method of claim 14, wherein Kd_{A}α₂>Kd_{B}α₂.

22. The method of claim 21, wherein Kd_{A}α₁>Kd_{B}α₁.

23. The method of claim 14, wherein the first aptamer α₁ is bound to the support at an Aptamer-1 site, and the second aptamer α₂ is bound to the support at an Aptamer-2 site.

24. The method of claim 23, wherein the Aptamer-1 site is <40 μm from the Aptamer-2 site.

25. The method of claim 14, wherein the targets are selected from the group consisting of proteins, peptides, carbohydrates, polysaccharides, glycoproteins, hormones, receptors, antigens, antibodies, viruses, substrates, metabolites, transition state analogs, cofactors, inhibitors, drugs, dyes, nutrients, and growth factors.

26. The method of claim 14, wherein the first aptamer α₁ is bound to a first solid support and the second aptamer α₂ is bound to a second solid support.
